(19) United States
(12) Patent Application Publication
(10) Pub. No.: US 2019/0069827 A1
(43) Pub. Date: Mar. 7, 2019

(54) SYSTEM, METHOD, ARTICLE, GARMENT AND APPARATUS FOR OBTAINING BODILY INFORMATION FROM MENSTRUAL FLUID

(71) Applicant: Adrianna Davies, San Francisco, CA (US)

(72) Inventor: Adrianna Davies, San Francisco, CA (US)

(21) Appl. No.: 16/123,443

(22) Filed: Sep. 6, 2018

Related U.S. Application Data
(60) Provisional application No. 62/554,874, filed on Sep. 6, 2017.

(51) Int. Cl. A61B 5/15; A61B 5/157; A61B 5/145; A61B 5/00; A41B 9/04; A61F 13/20

(52) U.S. Cl. CPC A61B 5/150045; A61B 5/157; A61B 5/14546; A61B 5/6804; A61F 2013/8473; A61F 13/202; A61B 5/4362; A61B 5/0002; A61B 5/4845; A41B 9/04

(57) ABSTRACT

Systems, methods, techniques, articles, garments and apparatuses for obtaining bodily information from menstrual fluid. A patch portion with a biosensor is placed within the garment or article in communication with the menstrual blood flow. The measured bodily information obtained from the menstruating woman is then transmitted either physically or electronically to a lab or others for further analysis.

### CROSS REFERENCE TO RELATED APPLICATION

The present invention is a non-provisional of U.S. Patent Application Ser. No. 62/554,874, entitled “SYSTEM, METHOD, TECHNIQUE, ARTICLE, GARMENT AND APPARATUS FOR OBTAINING BODILY INFORMATION FROM MENSTRUAL FLUID,” filed Sep. 6, 2017, the subject matter of which is incorporated by reference herein.

### FIELD OF THE INVENTION

The present invention is directed to improvements in the treatment of women, including the treatment of the unborn.

### BACKGROUND OF INVENTION

Researchers are increasingly looking to conduct long term population level studies to collect large data sets from which they hope to uncover novel tests for early detection of the presence of a variety of diseases, in particular, cancers.

These clinical trials are costly and necessitate that a large numbers of patients come on-site for diagnostic screening, as well as to deliver specimens of their bodily fluids that are then analyzed and stored under controls dictated by the study protocols for their present and future research purposes.

In longitudinal studies, it is difficult to ensure that study participants follow and comply with the full study requirements mandated by the study's protocol. Participant dropout rates are high throughout all stages of participation in the trial, and each dropout is costly to both the study sponsors as well as the clinical trial administrators who invest significant time to enroll each patient. As an example, recruitment cost aside, it takes at least 2 hours to screen a participant for eligibility in the study, then also to obtain their informed consent in order to complete their enrollment. Coordinators also need to gather medical history data, and many trials will require some baseline diagnostic screening tests to determine a participant's health data at the start of the study. The further into the study a participant drops out, the more costly they are to the trial as the time and money invested in them is written off with no value or return in terms of complete data sets.

For researchers to truly make use of advancements in DNA sequencing technology, it is necessary to collect massive amount of data to detect patterns in population, apply machine learning, find the networks of mutations responsible for disease, and then develop a solution or novel test method. The strength and accuracy of these predictive models depends on the size of the data sets.

However, it is not easy to persuade millions of people to give up their data for research purposes, even if people agree with and support the cause, it is unrealistic for most to be able to comply with the full study requirements needed to generate a strong valid data set. Participating in a study where you have to undergo screening, consenting, share your medical history, then also travel to the site to undergo testing and deliver specimens—this is a significant time and inconvenience for most. For these big data studies to actually produce new research findings, study participants would have to attend follow up testing and additional specimen collection visits involving multiple site visits and blood draws, over many years. Simply, it is very difficult, often invasive, costly, and inconvenient to obtain multiple samples overtime from an eligible study participant.

However, the use of menstrual blood provides an attractive opportunity to enable regular data collection over long periods of time for a variety of studies, involving not only female subjects but also their unborn children. Recent investigations at Stanford have conducted a cross-sectional evaluation of menstrual blood: a complex biological fluid composed of three separate bodily fluids: systemic blood, vaginal fluid, cells from the uterus and endometrial lining which is shed during menstruation, and cells from in utero fetuses. Studies have shown considerable correlation with systemic blood in addition to other elements found present. 385 unique proteins can be detected in menstrual blood with markers for a variety of disorders such as endometriosis, breast, cervical, and ovarian cancer, in addition to STDs such as chlamydia gonorrhea and high risk human papillomavirus (HPV) virus.

In addition to the aforementioned benefits for large-scale studies, there is a need for the monitoring and treatment of individual women using a technique and apparatus that are simple and easy to use. In this manner, the health and wellbeing of women can be efficiently monitored.

Additionally, in connection with the treatment of female patients who are pregnant, there is also a need for the monitoring and treatment of the in utero unborn using a technique and apparatus that are simple and easy to use. In this manner, the health and wellbeing of fetuses can be efficiently monitored, along with the mother. With the growing sophistication of testing capabilities, the ability to parse the cells and DNA and RNA of the unborn fetus (or fetuses) from the mother can easily be determined.

There is, therefore, a present need to provide a system, method, technique, article, garment and apparatus for obtaining bodily information from menstrual fluid according to the claims that provides an article and technique that easy obtains menstrual blood for biometric measurement.

### SUMMARY OF THE PRESENT INVENTION

The present invention is directed to a Smart underwear article, system, technique, apparatus and methodology that allows for major cost reduction to clinical trial orchestrators, higher participation rates, higher quality data obtained, and more frequent data collection, enabling both specific clinical trial goals as well as forming a platform for discovery of new findings that benefit mankind The present invention also offers individual women a technique and mechanism to easily monitor and collect data on their physical wellbeing, as well as the physical wellbeing of the unborn children using a non-invasive and safe technique.

### BRIEF DESCRIPTION OF THE DRAWING

While the specification concludes with claims particularly pointing out and distinctly claiming the subject matter that is regarded as forming the present invention, it is believed that the invention will be better understood from the following description taken in conjunction with the accompanying DRAWINGS, where like reference numerals designate like structural and other elements, in which:

The various FIGURES set forth in the text hereinbelow provide representative views of various aspects and features of a Smart underwear article, system, technique, apparatus and methodology, employing the principles of the present invention in exemplary configurations.

FIG. 1 of the DRAWINGS generally illustrates an article, garment or undergarment that may employ the principles of the present invention, such as a panty or other underwear garment;

FIG. 2 of the DRAWINGS generally illustrates the article shown in FIG. 1 on a human female, particularly a menstruating human female;

FIG. 3 of the DRAWINGS generally illustrates a portion of the article shown in FIGS. 1 and 2 that employs a patch and biosensor in a crotch portion thereof to measure the menstrual blood;

FIG. 4 of the DRAWINGS illustrates the patch and biosensor shown in FIG. 3 in an alternate embodiment, where the patch and the biosensor are removable;

FIG. 5 of the DRAWINGS generally illustrates a methodology for the transfer of the menstrual blood and the bodily information obtained therefrom using the removable patch shown in FIG. 4 via mail or other physical transfer; and

FIG. 6 of the DRAWINGS generally illustrates an alternate methodology for the transfer of bodily information about menstrual blood measurements via electronic or like transfers to associated devices in a local or global network.

### DETAILED DESCRIPTION OF THE PRESENT INVENTION

The present invention will now be described more fully hereinafter with reference to the accompanying DRAWINGS set forth herein, in which preferred embodiments of the invention are shown. It is, of course, understood that this invention may, however, be embodied in many different forms and should not be construed as limited to the embodiments set forth herein; rather, these embodiments are provided so that the disclosure will be thorough and complete, and will fully convey the scope of the invention to those skilled in the art. It is, therefore, to be understood that other embodiments can be utilized and structural changes can be made without departing from the scope of the present invention.

The principles of the present invention are directed to the development of an easy to use device, system, article, garment, and method for 1) controlled real time-data analysis of qualitative biomarkers measured from menstrual blood, the 2) utilization of the measurements obtained by the individual, and 3) subsequent storage of menstrual blood samples, such that sample specimens collected in the device can be biobanked for future use by or sale to pharma & biotech companies.

It should be understood that an intended use of the present invention is to provide a simple and straightforward mechanism for an individual female to assess their own health or the health of another loved one that menstruates, such as a drug-using daughter or ailing parent. The principles of the present invention can also be employed to assess the health of unborn children or fetuses through analysis of the mother's menstrual blood.

It should be understood that another intended use for the present invention is for clinical trial monitoring of women, particularly in multi-year longitudinal studies. For example, clinical trials studying diseases related to women's reproductive health and infectious diseases. Also, use in clinical trials studying hundreds of thousands to millions of patients simultaneously to see if there are the beginning signs of cancer emerging, or any abnormal cellular changes that may warrant further investigation by scientists.

It should also be understood that a primary implementation of the present invention is within a woman's undergarment, such as a panty, that is proximate to and has communication with vaginal or menstrual fluid flow. A representative garment or article or panty is shown in FIG. 1 of the DRAWINGS and generally designated by the reference numeral 100. It should be understood that alternate garments may be employed that utilize the principles of the present invention.

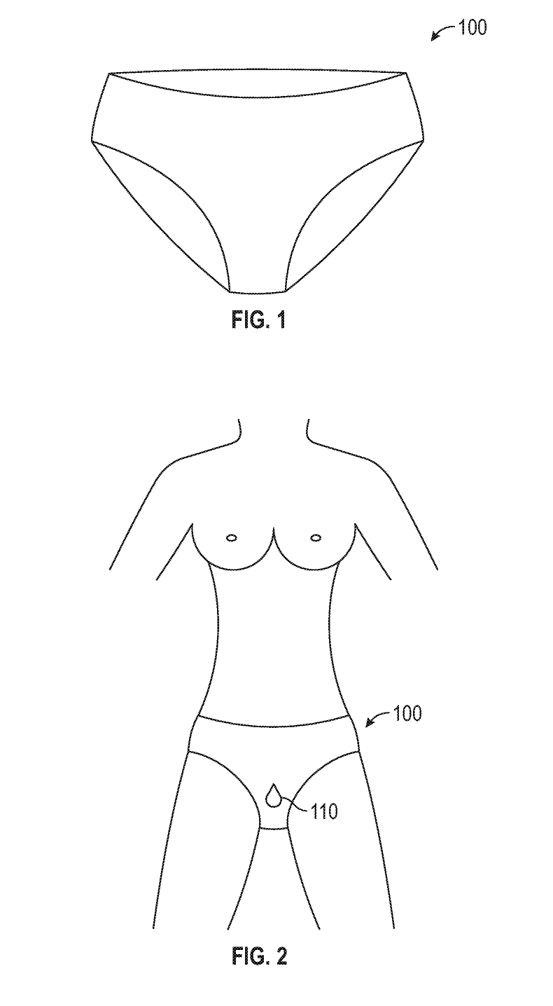

A normal use of the article 100 of FIG. 1 is shown in FIG. 2 of the DRAWINGS, which illustrates the intimate communication of the article 100 to the genital area of the woman shown, particularly to menstrual flow therefrom, such flow generally designated by the reference numeral 110.

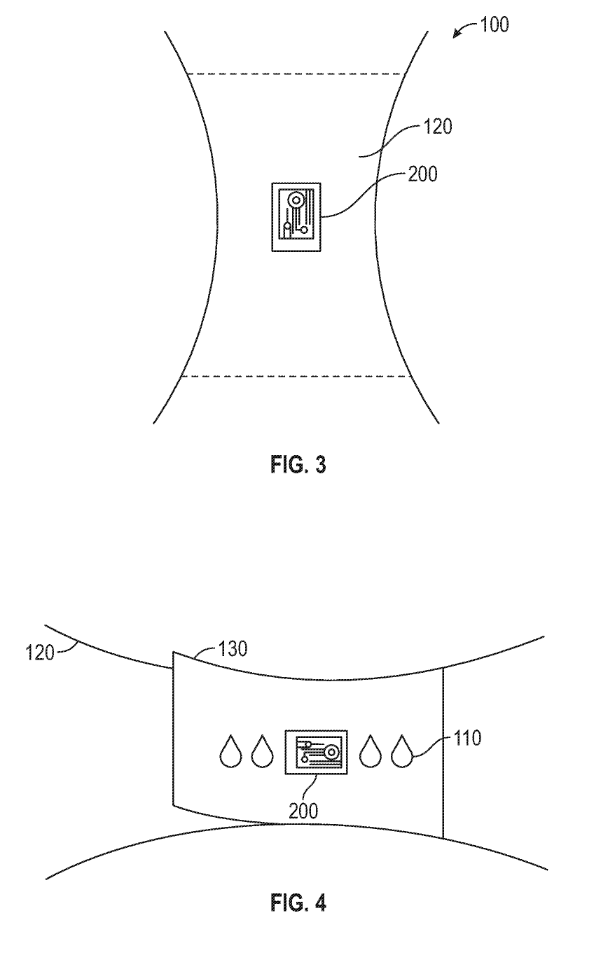

With reference now to FIG. 3 of the DRAWINGS, there is shown a view of the crotch portion of the article or panty 100, such as shown in FIGS. 1 and 2, the crotch portion thereof being generally designated herein by the reference numeral 120. In a preferred embodiment, the panty 100 has a removable patch, shown in more detail in FIG. 4, generally designated by the reference numeral 130, which is detachably affixed to the inside portion of the panty 100 along the crotch portion 120. It should be understood that the patch 130 is preferably contoured to fit the crotch area 120 with minimal or no discomfort to the subject woman.

It should, of course, be understood that the aforesaid menstrual fluid or flow 110 is onto said portion 120 and/or patch 130 or thereabout, which preferably absorbs at least a portion of the menstrual flow and channels the flow to the sensors, described hereinbelow. It should, of course, be understood that alternate embodiments are contemplated, e.g., an article 100 that is specially designed with the patch 130 built-in or integral with the panty 100 fabric or material, as shown in FIG. 3.

With further reference to FIGS. 3 and 4, the patch 3 preferably includes one or more or a plurality of sensors or biosensors, generally designated by the reference numeral 200, which collects a small portion of the channeled menstrual flow 110, and then measures and analyzes the menstrual blood or fluid 110 for various ailments or conditions. It should be understood that the biosensor 200 is preferably situated on the patch 130 in a position to receive the aforesaid menstrual flow 110, e.g., the patch 130 is preferably configured and positioned to channel at least a portion or sample of the menstrual flow 110 to the biosensor or biosensors 200.

It should, therefore, be understood that, in integral embodiments, the biosensor or biosensors 200 can be over the fabric of the article 100, i.e., the biosensor 200 is in direct contact with the genital area, under the fabric of the article 100, with the aforesaid flow 110 channeled through the fabric 100 to the biosensor 200, or integral thereto, as described in more detail herein.

As with the patch 130 being possibly integral with the article 100, i.e., the crotch portion 120 thereof, the biosensor 200 can be integral with the patch 130, or in a currently-preferred embodiment be affixed thereto, such as with Velcro or other securement means as it understood in the art. As the patch 130 and/or the biosensor 400 may require cleaning and/or replacement, the present invention contemplates that the patch 130 and biosensor 400, whether integral or not, can be easily removed for cleaning or replacement.

It should, of course, be understood that in another preferred embodiment, the patch 130, with or without the biosensor 400 thereon, may be disposable after one or a small number of uses, or be sufficiently resilient for ongoing usage, which would entail that the patch 130 materials and the biosensor 400 equipment be configured to withstand washing or cleaning.

With particular reference now to FIG. 4 of the DRAWINGS, there is shown the aforementioned patch 130 placement as in FIG. 3, but better illustrating the removability of the patch 130 in a preferred embodiment from the article 100, enabling better access to the biosensor 200 thereon, which can also be detached from the patch 130 for processing.

It should be understood that the patch 130 should be made of a material that can absorb the menstrual blood 110, and not only hold the fluid 110 therein but also channel some to the biosensor 200, as described hereinabove. The patch 130 material is thus preferably an absorbent material, preferably layered, and proximate to the vaginal blood flow 110. It should also be understood that the patch 130 and biosensor 200 can also couple with a feminine hygiene product, such as a tampon. It should thus be understood in a further embodiment that the patch 130 material itself can act as a tampon, i.e., provide absorbency of the menstrual fluid beyond what is needed for the biosensors 200.

Further to the above, in an alternate embodiment of the present invention, the biosensor 200 is operatively connected to the patch 130, and can be an electrochemical detector, and can include lateral flow assaying of the detected and collected blood, whether at the onset of menses, toward the end of menses or at any points therebetween.

The biosensor 200 may have an annunciation means to indicate its receipt of blood measurement data or indicate its readiness to convey or transmit that information, which may be covert or non-covert in view of privacy concerns. Alternatively, and preferably, the biosensor 200 stores the results obtained from the menstrual flow 110 in a memory portion therein, where the biosensor and its memory can be physically removed and transferred, as described in more detail hereinbelow.

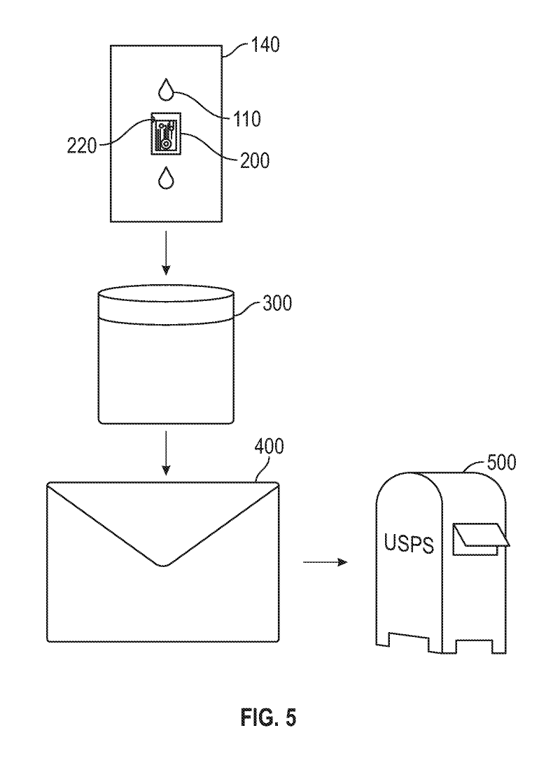

With reference now to FIG. 5 of the DRAWINGS, there is shown a currently-preferred embodiment of the present invention where the patch 130 and the biosensor 200 are detached from the article 100, along with the bodily information or measurements therein, now stored therein within the biosensor 200 memory, as well as within the absorbent layers of the patch 130, i.e., the blood 110 soaked in the patch 130 material.

In this embodiment there is illustrated a removed patch, generally designated by the reference numeral 140, i.e., a patch 140 with the biosensor 200 that has been detached from the crotch portion 120 of the panty article 100. As with some portable testing techniques, the blood 110 within the patch 140 material and within the biosensor 200, can be forwarded to a remote place, i.e., the patch 140 can be detached, as illustrated in FIG. 5, placed in a pouch or other sealed container, generally designated by the reference numeral 300, in an effort to minimize biodegradability during transit. The pouch 300, with the patch 140 and biosensor 200 therein, is then placed in an envelope, box or other container, generally designated by the reference numeral 400, such as for mailing through a postal system, such as U.S. mail, generally designated by the reference numeral 500, for delivery elsewhere, e.g., a lab.

In this embodiment, with the removable patch 140 with the sensor 200 thereon, it should be understood that the patch 140 can be cleaned and reused, as described, with the sensor 200 detached therefrom and mailed. Alternatively, the entire patch 140 with the sensor 200 can be mailed. In still another alternative embodiment, the entire undergarment 100 with the patch 140 (or with the patch 130 integral with the crotch portion 120) and the sensor 200 can be mailed.

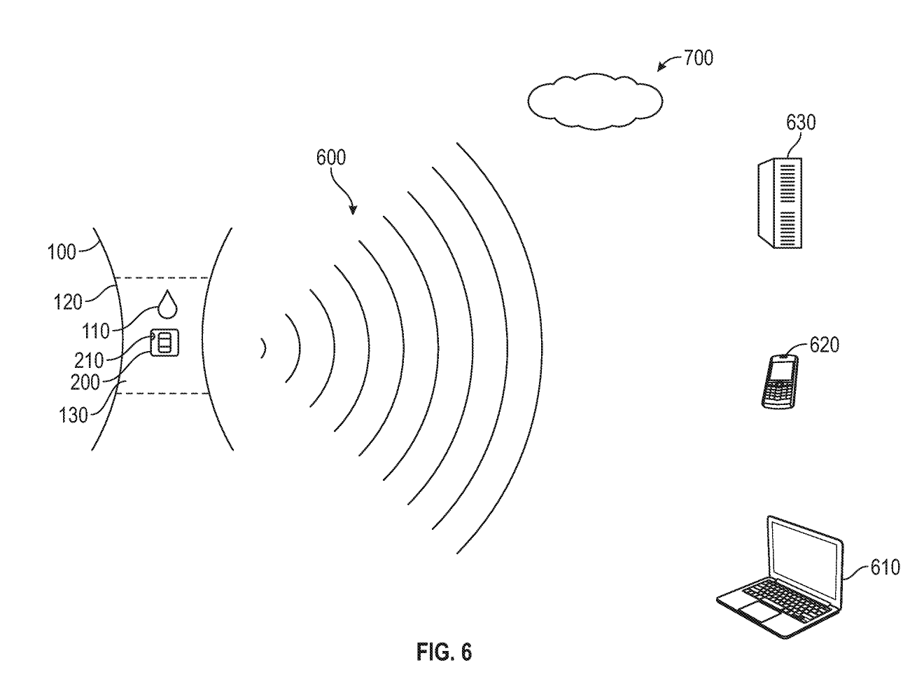

With reference now to FIG. 6 of the DRAWINGS, there is shown another embodiment for processing the obtained information detected by the biosensor 200. Instead of physically mailing the results obtained from measuring the blood 110, as described in connection with FIG. 5, the testing results can instead be electronically transmitted, generally designated by the reference numeral 600. As shown in FIG. 6, the biosensor 200 on the patch 130 receives the menstrual blood or fluid 110, measures the constituent parts thereof, and then radiates that information outward for remote reception.

It should be understood that the biosensor 200 can store measurement readings for some time and then transmit, e.g., using a transmitter therein, generally designated by the reference numeral 210, at the onset of menses, at the end of menses, or at any points therebetween, as described hereinabove. Preferably, the transmitter 210 is privacy protected and operable only as directed by the woman.

It should be understood that the measurements of the biosensor 200 can be transmitted 600 via a local signal or limited-range transmission to a device. For example, the data can be wirelessly transmitted to a local or proximate computer or laptop, generally designated by the reference numeral 610, e.g., the measurements can be converted to a transmission formal such as ultra-wide band (UWB) or other format, transmitted by the biosensor 200, received at a receiver in the PC 610 and processed. Likewise, the transmission to a smart phone of other portable device, generally designated by the reference numeral 620, or to a local or remote server, generally designated by the reference numeral 630, as is understood to one of skill in the art.

It should further be understood that the sensor or biosensor 200 may transmit the aforesaid bodily information automatically, upon completion of the aforementioned analysis, episodically, e.g., hourly, daily, weekly, monthly, etc., or pursuant to another timetable. It should be understood that the biosensor 200 may be passive, i.e., only transmitting upon the receipt of an external command for downloading of the bodily information.

A processor within the devices 610, 620 and 630 can process the transmitted measurements and display them in a readable output, e.g., on the display of the PC 610 or the phone 620, or a remote device, such as at a lab or doctor's office, easily relaying pertinent and dynamic information regarding the health or wellbeing of a patient.

As discussed, it should be understood that the biosensor 200 can transmit the aforesaid measurements of a given user or patient automatically, periodically or per a timetable to the devices 610, 620 and 630. Additionally, any of the devices 610, 620 and 630 can remotely monitor the biosensor 200 and dynamically request the health measurements at any time, and download the bodily information.

The transmitted information 600 may also be passed to the so-called cloud, generally designated by the reference numeral 700, for remote storage, where another device (or remote devices) connected thereto can access and obtain the data within the biosensor 200. As discussed, it should be understood that due to privacy concerns, encryption of other protective means should be employed to protect against intrusion and demand verification before access. It should be understood that the biosensor 200 can communicate data through a number of technologies: wireless signals 700, Bluetooth, RF frequency, and other means to the cloud 700, or directly to servers 630 and web/mobile devices 610 and 620, as described herein.

In one currently-preferred embodiment, the crotch portion 120 of the article 100 has a biosensor 200 embedded in a removable patch layer 130, where the patch 130 is made of sterile deionized cotton (similar to materials used in forensic swabs). The biosensor 200 is preferably able to make qualitative determinations of the presence of infectious diseases (and other ailments) in ‘real-time,’ e.g., roughly 120 seconds, such as for immediate transmission elsewhere as described hereinabove, or for ease of use in a lab downloading biosensors received in the mail, as described in connection with FIG. 5.

In another embodiment of the present invention, the crotch portion 120 of the underwear garment 100 is a removable patch 130 made of a unique absorbent material designed for DNA and/or RNA capture (similar to swabs used in forensics). The patch 130 may be treated with a buffer in order to enhance the stability of the sample for long term storage, and future DNA amplification and extraction in a lab, biobank, or clinical research site, such as used in the mailing protocol shown in FIG. 5 or storage.

In yet another embodiment of the present invention, the smart panty 100 can be developed as a reusable form factor. A box, such as box or container 300, for sale or use by a patient or subject would include one SmartStrand underwear garment with washable Velcro attachments in the crotch base 120 of the underwear 100. The kit or box 300 would also include perhaps twelve removable crotch patches 130 for use once per month during the study participants menstrual cycle, and perhaps a corresponding twelve multi-barrier pouches containing desiccant and associated pre-paid postage labels for use, e.g., with the embodiment described in connection with FIG. 5.

It is, of course, an intended use of the present invention is clinical trial monitoring of women, particularly in multi-year longitudinal studies, but, as shown, there are also significant personal advantages to individual women, such as with the commercial use of the techniques and apparatuses described herein.

In connection with the clinical trial embodiment of the present invention, mass testing, considering a clinical trial would consist of multiple study participants, ranging from as small as (50-1,000,0000 participants), a study sponsor can order the SmartStrand kits prepared with appropriate prepaid and addressed postage labels. The labels on the outside of the box preferably has customizable labels that include form fields for: the study sponsor name, clinical trial site location, study coordinator contact details, Study ID, and study participant ID.

After the box contents have been used by a study participant, the clinical trial coordinator would have monthly real-time data delivered in 120 seconds from contact with a few drops of menstrual fluid 110 with the biosensor 200 embedded in the removable crotch piece 130 and measuring qualitative biomarkers. In addition, they would have DNA collected and preserved each month from that study participant on the removed crotch piece or patch 140. The patches 130/140 can be immediately analyzed in a lab with standard DNA/RNA extraction techniques.

The DNA can undergo any number of manipulations such as sequencing, therapy, and gene editing applications using CRISPR technology. Recently, Scientists Dr. James J. Collins and Dr. Feng Zhang of the Broad Institute of MIT and Harvard developed a CRISPR-based tool that can detect tiny amounts of Zika and Dengue virus, distinguish pathogenic bacteria, and identify DNA variations. The components of the system can be freeze-dried and reconstituted on paper for field applications, opening the door to many practical uses. There is, and will continue to be, an increasing number of clinical trials conducting research on different aspects of leveraging CRISPR and CRISPR enabled diagnostic tests to treat and prevent diseases and genetic disorders. The SmartStrand Garment Technology will enable this kind research as it effectively collects and preserves valuable data and DNA samples on the removable patch 130 in a fashion unprecedented until now.

The patches 130 can be analyzed immediately and then stored in a biobank for analysis downstream. The patches 130 can also be stored immediately in a biobank once received by the clinical trial coordinator without immediately extracting DNA samples. This will depend on the specific needs of the clinical trial using the SmartStrand Garments process, and the process is flexible to the needs of various study protocols, and perhaps for other purposes, such as in criminal or other legal proceedings.

The smart panty invention capitalizes on a normal, recurrent excretion of a bodily fluid by women, and a normal behavior requiring no behavioral changes: the use of an undergarment or ‘panty’. The device, article or garment 100 can capture data in a controlled fashion with little difficulty or friction for study participants, thereby increasing participation rates so a critical mass of data can more easily and cheaply be obtained and exploited. This data will be stored on a secure searchable platform that can be mined by researchers, institutions, and companies for a cost.

The smart panty 100 of the instant invention thus enables a broad spectrum of participant data to be captured each month during the participant's menstrual cycle, i.e., from the menstrual fluid 110. The device or article 100 requires no change in normal behavior other than refraining from use of any kind of menstrual hygiene product while they wear the smart panty 100. The device 100 can be worn when a patient is off-site and conducting their normal daily activities for example at work, school, or caring for the home and household.

Another advantage of the present invention is that this convenience will perhaps reduce the aforementioned difficulty or friction in complying with study requirements, as participants do not need to travel to the site to deliver specimens or undergo painful blood draws. By wearing the smart panty 100, they first produce monthly real-time data points for the investigator, then the removable patch 140 on the crotch portion 120 of the panty 100, is placed in a special multi-barrier pouch 300 and mailed 500 by normal mail to the study site or biobank for storage in a condition where it can be recovered and analyzed in the future, such as described in more detail in connection with the embodiments shown and described in FIG. 5.

There are several advantages of the present invention, including the simple fact that the articles of the present invention, such as Smart String panties 100, may be used for blood testing every month, checking for the presence of qualitative markers over time for a variety of markers.

Another advantage is that real-time data that can be gathered by the smart panties 100 of the present invention, enabling the ability to accurately detect the presence (or absence) of infectious diseases, such as sexually transmitted infections, Chikungunya and Zika, for example. In other words, the present invention can test for any qualitative biomarker.

A further advantage is that there are very few limits to the data obtainable from the removable patch 140 from the crotch portion 120, which may be mailed 500 to a testing site for analysis and or storage in a biobank. The highest standard of protocol for DNA preservation for storage and future research will be used with the specimens stored in the mailed-in crotch patch. Any marker currently analyzable with standard laboratory methods for DNA can potentially be examined with the smart panty device 100.

An additional and important usage of the principles of the present invention involves the analysis of the fluid 110 not just for the subject, but for her unborn fetus. Mothers may want an easy and effective way to obtain non-invasive, prenatal fetal cells, which would be present in her won menstrual fluid 110, and detectable pursuant to the teachings of the present invention, as described hereinabove and hereinbelow. The various mechanisms for the testing and sharing of the information are as set forth hereinabove. It is, therefore, apparent that the techniques of the present invention, set forth in more detail hereinabove and hereinbelow, are directly applicable to both the mother and unborn child.

In addition to all of the usages discussed hereinabove, the articles 100 and techniques of the present invention are readily applicable to generally or specifically monitor mothers during their pregnancy, e.g., for the presence of disease and cancer, as above, but also for drugs and harmful substances, whether to the fetus or also the mother.

It should also be understood that although the principle use of the present invention is to use menstrual fluid, other types of bodily discharge may be employed, as discussed, and are envisioned.

With the radiofrequency embodiment of the instant invention, as set forth in FIG. 6, mothers that have a harmful chemistry, as determined from their menstrual fluid 110 and other discharges, can be alerted, or others alerted, as discussed. Accordingly, another usage of the instant invention, although perhaps easily circumvented, is this dynamic monitoring of the subject, primarily in menses but in another embodiment of the present invention, additional times also through the capture and analysis of other discharges.

The previous descriptions are of preferred embodiments for implementing the invention, and the scope of the invention should not necessarily be limited by these descriptions. It should be understood that all articles, references and citations recited herein are expressly incorporated by reference in their entirety. The scope of the current invention is defined by the following claims.

What is claimed is:

1. An undergarment comprising:
a crotch portion;
a patch, said patch being affixed to said crotch portion of said undergarment and configured to be in contact with menstrual flow; and
at least one sensor, said at least one sensor being positioned on said patch and configured for communication with said menstrual flow,
wherein said at least one sensor, configured to analyze menstrual fluid upon communication with said menstrual flow, analyzes said menstrual fluid and determines bodily information therefrom.

2. The undergarment according to claim 1, wherein said at least one sensor comprises a plurality of biosensors.

3. The undergarment according to claim 1, wherein said at least one sensor comprises a memory, said memory storing said bodily information from said menstrual fluid.

4. The undergarment according to claim 3, wherein said at least one sensor is configured to perform a lateral flow assay of said menstrual fluid.

5. The undergarment according to claim 3, wherein said at least one sensor further comprises an annunciator.

6. The undergarment according to claim 1, wherein said at least one sensor analyzes said bodily information for a body condition, said body condition selected from the group consisting of cancers, infectious diseases, genetic disorders, sexually-transmitted diseases, drug use, and combinations thereof.

7. The undergarment according to claim 6, wherein said at least one sensor senses for at least one DNA or RNA marker.

8. The undergarment according to claim 1, wherein said bodily information from said menstrual fluid includes bodily information for an unborn child.

9. The undergarment according to claim 1, wherein said patch is detachable from said undergarment.

10. The undergarment according to claim 9, wherein said patch affixes to said crotch portion of said undergarment using Velcro material.

11. The undergarment according to claim 9, wherein said patch is made of a cleanable material.

12. The undergarment according to claim 9, wherein said patch is made of sterile deionized cotton.

13. The undergarment according to claim 9, wherein said patch is configured within a tampon.

14. The undergarment according to claim 1, wherein said patch is integral to said undergarment.

15. The undergarment according to claim 1, further comprising:
a transmitter, said transmitter transmitting said bodily information.

16. A system for obtaining bodily information from menstrual fluid comprising:
an article, said article being worn by a menstruating woman;
a patch, said patch being affixed to a crotch portion of said article;
at least one biosensor, said at least one biosensor positioned on said patch and in communication with menstrual fluid,
wherein said at least one biosensor, configured to analyze said menstrual fluid upon communication with said menstrual flow, analyzes said menstrual fluid and determines bodily information therefrom;
a transmitter, said transmitter transmitting said bodily information from said at least one biosensor,
whereby bodily information about said menstruating woman can be obtained with minimal intrusion.

17. The system according to claim 16, wherein said patch is detachable from said article, and wherein said patch and said at least one biosensor are placed in a pouch and mailed to a lab.

18. The system according to claim 17, wherein said detachable patch comprises a reusable form factor.

19. The system according to claim 16, wherein said bodily information from said menstrual fluid includes bodily information for an unborn child.

20. The system according to claim 16, wherein said transmitter transmits said bodily information to another device, said another device elected from the group consisting of computers, laptops, servers, smart phones, a cloud and combinations thereof.

21. A method for obtaining and analyzing bodily information from menstrual fluid comprising:
positioning at least one biosensor on a patch, said patch being affixed to the crotch portion of an undergarment for a woman, said at least one biosensor thereon configured for communication with menstrual fluid from said woman,
obtaining, upon menstruation by said woman, a sample of said menstrual fluid from said woman;
analyzing, by said at least one biosensor, said sample, and determining bodily information therefrom; and
transmitting said bodily information.

22. The method according to claim 21, wherein said bodily information from said menstrual fluid includes bodily information for an unborn child.
